(19) United States
(12) Patent Application Publication
(10) Pub. No.: US 2010/0261266 A1
(43) Pub. Date: Oct. 14, 2010

(54) BIOLOGICAL DESULFURIZATION APPARATUS

(76) Inventors: Yasuhiko Nagamori, Fuchu-shi (JP); Takumi Obara, Fuchu-shi (JP); Nobuyuki Ashikaga, Kawasaki-shi (JP); Hiroshi Tamura, Tokyo (JP); Takayuki Ishige, Yokohama-shi (JP); Takeo Yamamori, Kawasaki-shi (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/527,700

(22) PCT Filed: Dec. 19, 2008
(86) PCT No.: PCT/JP2008/073880

§ 371 Date: Aug. 19, 2009

(30) Foreign Application Priority Data
Dec. 28, 2007 (JP) 2007-339948
Feb. 14, 2008 (JP) 2008-033319
Feb. 18, 2008 (JP) 2008-036264

(51) Int. Cl. C12M 1/107

(52) U.S. Cl. 435/300.1

(57) ABSTRACT

Disclosed is a biological desulfurization apparatus including a reaction tower 1 into which biogas generated by anaerobic fermentation of organic waste is to be introduced, a carrier-packed layer 2a, 2b arranged in the reaction tower and provided with a carrier to which microorganisms have adhered, an air feeding pipe 3 that mixes air into a lower part of the reaction tower, and a sprinkling mechanism 7 that sprinkles water on an upper part of the reaction tower, wherein the carrier-packed layer is arranged in two or more stages.

### TECHNICAL FIELD

The present invention relates to a biological desulfurization apparatus, and in particular to a biological desulfurization apparatus for biogas generated by anaerobic digestive treatment of organic discharged water such as sewage and industrial drainage.

### BACKGROUND ART

A methane fermentation process is often used as a method of treating organic waste such as sewage sludge and raw garbage or organic discharged water such as food factory effluent. The methane fermentation process is a treatment process wherein organic discharged water is introduced into a reaction tower and organic matter in the discharged water is decomposed by a group of methane fermentation bacteria packed in a reaction tank, thereby forming biogas consisting primarily of methane gas and simultaneously decomposing and removing the organic matter. However, when sulfur components such as those derived from proteins are contained in discharged water, the sulfur components are reduced by the action of sulfate reducing bacteria, and thus the resulting biogas contains hydrogen sulfide gas.

When methane gas contained in biogas is used as a fuel in a boiler, etc., the hydrogen sulfide gas contained in biogas should be removed. This is because the hydrogen sulfide gas in biogas is oxidized upon combustion of biogas, and forms sulfur oxide which can in turn corrode facilities.

The method used in removing the hydrogen sulfide gas contained in biogas includes a dry-desulfurization method of removing the gas by adsorption onto an adsorbent consisting primarily of iron oxide, and a wet-desulfurization method of removing the gas by absorption into an aqueous solution using an alkali, etc. However, these methods are used in a system where running costs are rocketed because chemicals such as an adsorbent are required for adsorption and the adsorbent after adsorption turns to waste.

A technique which comprises charging a reaction tank with a carrier to which microorganisms for oxidative decomposing hydrogen sulfide adhere, thereby removing hydrogen sulfide in biogas (Jon. Pat. Apple. KOKI Publication No. 2-26615) has been proposed as a system of desulfurization with low running costs.

In this desulfurization technique, there has been pointed out a problem that a packed layer is easily clogged with microorganisms multiplied at the bottom of the packed layer or on a supporting part of the packed layer or with a sulfur component formed by oxidization of the removed hydrogen sulfide. As a means of solving this problem, a washing method has been proposed in which biogas is flowed as a downward stream, while a carrier lighter than water is used and allowed, by blowing air from the bottom, to float in water filled to a level higher than the packed layer (Japanese Patent No. 3750648).

For removing hydrogen sulfide gas contained in biogas by sulfur-oxidizing bacteria, a sufficient amount of the microorganisms should be held on a carrier in the packed layer. However, when a sulfur component deposited in the packed layer is removed by washing with water, the microorganisms may also be washed away together with a sulfur component. That is, when the washing time is short, a sulfur component cannot be sufficiently removed, thus easily permitting clogging. On the other hand, when the washing time is long, a sufficient amount of microorganisms necessary for removal of hydrogen sulfide cannot be secured, so there arises a problem that the ability to remove hydrogen sulfide is reduced after washing. During washing, the carrier is allowed to float in water, thus increasing the efficiency of washing, but because the supporting plate for the carrier is fixed, its adhering matter is not sufficiently removed by washing and may allow the carrier supporting plate to be clogged to cause a problem of reduction in treatment efficiency.

When the biological packed layer is increased in height, oxygen in the air mixed in the lower part of the packed layer is consumed. Accordingly, when the amount of mixed air is small, there occurs the case where microorganisms in the upper part of the packed layer cannot sufficiently flourish.

### DISCLOSURE OF INVENTION

An object of the present invention is to provide a biological desulfurization apparatus for biogas, which can prevent clogging in a carrier-packed layer, can hold microorganisms adhering to a carrier, and can supply necessary oxygen to the packed layer, thereby enabling stable treatment.

A biological desulfurization apparatus according to an aspect of the present invention comprises a reaction tower into which biogas generated by anaerobic fermentation of organic waste is to be introduced, a carrier-packed layer arranged in the reaction tower and provided with a carrier to which microorganisms have adhered, an air feeder that mixes air into a lower part of the reaction tower, and a sprinkling mechanism that sprinkles water on an upper part of the reaction tower, wherein the carrier-packed layer is arranged in two or more stages.

According to the present invention, there can be provided a biological desulfurization apparatus for biogas, which can prevent clogging in the carrier-packed layer and can hold microorganisms adhering to the carrier, thereby enabling stable treatment.

### BRIEF DESCRIPTION OF DRAWINGS

FIG. 1 is a skeleton framework of the biogas biological desulfurization apparatus of the present invention in Example 1.

FIG. 2 is a skeleton framework of the biogas biological desulfurization apparatus of the present invention in Example 2.

FIG. 3 is a skeleton framework of the biogas biological desulfurization apparatus of the present invention in Example 3.

FIG. 4 is a schematic explanatory diagram of the biological desulfurization apparatus of the present invention in Example 4.

FIG. 5A is a schematic plane view of a supporting plate for the upper-stage packed layer in one constitution of the biological desulfurization apparatus of the present invention in Example 5.

FIG. 5B is a schematic plane view showing the relationship between pipes and valve or a blower in one constitution of the upper-stage aeration unit arranged below the upper-stage packed layer.

FIG. 5C is an enlarged partial back-side view of the aeration pipe in FIG. 5B.

FIG. 6 is a schematic overview of the biological desulfurization apparatus of the present invention in Example 7.

FIG. 7A is a schematic explanatory diagram of the biological desulfurization apparatus in Example 8.

FIG. 7B is a characteristic curve showing the relationship between the tower height of the water level in the reaction tower of the biological desulfurization apparatus and the temperature of the external surface of the tower.

FIG. 8 is a schematic explanatory diagram of the biological desulfurization apparatus of the present invention in Example 9.

FIG. 9 is an explanatory diagram of the biological desulfurization apparatus of the present invention in Example 10.

FIG. 10 is an explanatory diagram of the biological desulfurization apparatus of the present invention in Example 11.

FIG. 11 is an explanatory diagram of the biological desulfurization apparatus of the present invention in Example 12.

FIG. 12 is an explanatory diagram of the biological desulfurization apparatus of the present invention in Example 13.

FIG. 13 is an explanatory diagram of the biological desulfurization apparatus of the present invention in Example 14.

FIG. 14 is an explanatory diagram of the biological desulfurization apparatus of the present invention in Example 15.

### BEST MODE FOR CARRYING OUT THE INVENTION

The biological desulfurization apparatus in a first aspect of the invention comprises a reaction tower into which biogas generated by anaerobic fermentation of organic waste is to be introduced, a carrier-packed layer arranged in the reaction tower and provided with a carrier to which microorganisms have adhered, an air feeder that mixes air into a lower part of the reaction tower, and a sprinkling mechanism that sprinkles water on an upper part of the reaction tower, wherein the carrier-packed layer is arranged in two or more stages.

The amounts of multiplied microorganisms and formed sulfur components that are the cause of clogging in the packed layer are proportional to the amount of hydrogen sulfide removed and are thus increased in the lower part of the packed layer at the biogas-inflow side where the hydrogen sulfide concentration of biogas is high, or in that site in the packed layer where the flow rate of biogas is high. Once such site is clogged, a site adjacent thereto is then subjected to an increased flow rate of biogas and thereby clogged. That is, when the packed layer is a continuing layer, clogging spreads continuously from one site to another site. Accordingly, the packed layer is divided into packed layers, to provide the apparatus with a plurality of packed layers, thereby allowing spaces among the packed layers to serve as gas buffers with which the disproportional flow rate of biogas is lessened. Clogging in the packed layer is thereby prevented and the ability of the apparatus to remove hydrogen sulfide is stabilized.

In the first aspect of the invention, the diameter of the carrier in the carrier-packed layer at the lower-stage side is preferably larger than the diameter of the carrier in the carrier-packed layer at the upper-stage side. By increasing gaps in the carrier-packed layer at the lower-stage side in this manner, the period up to clogging can be prolonged. Thus by reducing the frequency of washing, the amount of microorganisms adhering to the carrier-packed layer at the lower-stage side can be increased to improve the efficiency of treatment.

In the first aspect of the invention, an air pipe for washing is arranged preferably in each of the carrier-packed layers, thereby constituting the apparatus such that the carrier-packed layers can be washed separately from one another. The lower part where solids are easily accumulated, or the upper part, can thereby solely be washed. Accordingly, the reaction tower can be prevented from clogging, and the clogged carrier-packed layer can solely be washed, thereby preventing the deterioration in treatment performance caused by a deficiency of microorganisms after washing.

In the first aspect of the invention, a sprinkling mechanism is arranged in every carrier-packed layer or in every predetermined number of carrier-packed layers. That is, if a sprinkling mechanism is arranged in only the upper part of the reaction tower, there can be a case where, when the flow rate of the sprinkling mechanism is increased, which prevents accumulation of solids in the carrier-packed layer at the lower-stage side, microorganisms that have adhered to the surface of the carrier-packed layer at the upper-stage side are also allowed to flow. Hence, a reduction in the amount of the microorganisms in the reaction tower is caused, and the performance of the reaction tower in biological desulfurization may be deteriorated. However, when a sprinkling mechanism constituted as described above is arranged separately from the sprinkling mechanism at the upper-stage side, solids accumulated in the carrier-packed layer at the lower-stage side can be removed by washing. The sprinkling mechanism can be arranged in every predetermined number of carrier-packed layers; for example, when 2 carrier-packed layers are arranged, the sprinkling mechanism can be arranged between the upper and lower carrier-packed layers, or when 6 carrier-packed layers are arranged, the sprinkling mechanism can be arranged, for example, every two layers. However, the arrangement of the sprinkling mechanism is not limited to these examples.

In the first aspect of the invention, a sprinkling mechanism (for example, a sprinkling nozzle) other than that in the uppermost stage is preferably directed upward. A sprinkling mechanism other than the sprinkling mechanism at the upper-stage side, when arranged for example between the upper and lower carrier-packed layers and directed upward, will shower water directly on the carrier-packed layer at the upper-stage side. As a result, a supporting plate of this carrier-packed layer can always be washed to prevent a sulfur component from adhering to the supporting plate, thereby preventing the carrier-packed layer from clogging.

In the first aspect of the invention, the amount of water to be sprinkled from the intermediate sprinkling mechanism is preferably larger than that from the upper sprinkling mechanism. As described above, clogging in the packed layer occurs more easily in a portion nearer to the side of biogas inflow. Thus, by increasing the amount of water to be sprinkled from the sprinkling mechanism nearer to the side of biogas inflow, clogging in the packed layer can be prevented, while unnecessary removal of microorganisms does not occur, thus preventing deterioration in the ability of the biological desulfurization apparatus to remove hydrogen sulfide.

In the first aspect of the invention, a plurality of carrier-packed layers are further provided in an intermediate portion with an intermediate air feeder of introducing air. By such constitution, oxygen can be supplied to microorganisms that have adhered to the surface of the carrier-packed layer at the lower-stage side, even when the concentration of hydrogen sulfide contained in influent biogas fluctuates or when the amount of microorganisms that have adhered to the carrier-packed layer at the upper-stage side is increased. Accordingly, the activity of the microorganisms can be maintained, and even if the concentration of hydrogen sulfide in influent biogas fluctuates, the efficiency of treatment can be stabilized.

According to the first aspect of the invention, clogging in the carrier-packed layer can be prevented, and simultaneously microorganisms that have adhered to the carrier can be maintained, thereby enabling stable treatment.

The biological desulfurization apparatus in a second aspect of the invention is a biological desulfurization apparatus comprising a reaction tower provided at the upper part thereof with a lid or valve capable of opening and closing, upper- and lower-stage packed layers arranged vertically in the reaction tower respectively and consisting of a carrier and a supporting plate for supporting the carrier, upper- and lower-stage aeration units arranged on the undersides of the upper- and lower-stage packed layers respectively, and a treated-water pipe connected to the lower part of the reaction tower and having a drain valve interposed therein, wherein the lid or valve is opened, the drain valve is closed, the upper-stage packed layer is filled with water above the upper-stage packed layer and washed in this state with aeration by feeding air to the upper-stage aeration unit, and thereafter, the upper-stage aeration unit is stopped, the drain valve is opened, the water level is made lower than the upper-stage packed layer and higher than the lower-stage packed layer, and in this state, the lower-stage packed layer is washed with aeration by feeding air to the lower-stage aeration unit.

The biological desulfurization apparatus in a third aspect of the invention is a biological desulfurization apparatus comprising a reaction tower provided at the upper part thereof with a lid or valve capable of opening and closing, upper- and lower-stage packed layers arranged vertically in the reaction tower respectively and consisting of a carrier and a supporting plate for supporting the carrier, upper- and lower-stage aeration units arranged on the undersides of the upper- and lower-stage packed layers respectively, and a treated-water pipe connected to the lower part of the reaction tower and having a drain valve interposed therein, wherein the lid or valve is opened, the drain valve is closed, and the supporting plate of the upper- or lower-stage packed layer is filled with, and immersed in, water above the supporting plate of the upper- or lower-stage packed layer and washed with aeration by feeding air to the upper- or lower-stage aeration unit.

The biological desulfurization apparatus in a fourth aspect of the invention is a biological desulfurization apparatus comprising a reaction tower, upper- and lower-stage packed layers arranged vertically in the reaction tower, a water-sprinkling shower nozzle arranged in the upper part of the reaction tower, a circulation unit that sprinkles, from the upper part of the upper-stage packed layer, a part of bottom accumulated water dropped via the upper- and lower-stage packed layers to the bottom of the reaction tower and accumulated at the bottom, and a treated-water pipe connected to the lower part of the reaction tower and having a drain valve interposed therein, wherein when microorganisms in the upper-stage packed layer are removed excessively by washing, the amount of the microorganisms in the upper-stage packed layer is recovered by simultaneous use of circulation of the bottom accumulated water by the circulation unit and water sprinkling by the shower nozzle.

The biological desulfurization apparatus in a fifth aspect of the invention is a biological desulfurization apparatus comprising a reaction tower provided at the upper part thereof with a lid or valve capable of opening and closing, upper- and lower-stage packed layers arranged vertically in the reaction tower and consisting of a carrier and a supporting plate for supporting the carrier, a high-pressure washing unit having a high-pressure washing nozzle, arranged respectively on the downsides of the upper- and lower-stage packed layers, and a treated-water pipe connected to the lower part of the reaction tower and having a drain valve interposed therein, wherein the upper- and lower-stage packed layers are washed by feeding water to the high-pressure washing nozzle of the high-pressure washing unit.

The apparatus in the second aspect of the invention is constituted preferably by comprising a means of washing the upper and lower-stage packed layers, and a means of washing the lower-stage packed layer in which the lid or valve is opened, the drain valve is closed, and the lower-stage packed layer is filled with water above the lower-stage packed layer and washed in this state with aeration by feeding air to the lower-stage aeration unit, wherein the frequency of washing of the lower-stage packed layer is carried out at shorter intervals than the frequency of washing of the upper-stage packed layer.

The reason for adoption of such constitution is as follows: That is, hydrogen sulfide is changed to sulfuric acid or a sulfur component by desulfurization reaction. Sulfuric acid is discharged together with discharged water, but a sulfur component adheres to the carrier and causes clogging in the upper-stage packed layer and lower-stage packed layer. However, it was known from previous research results that a sulfur component is formed in a larger amount in the lower-stage packed layer, and thus when washing of the lower-stage packed layer is conducted at a higher frequency than washing of the upper-stage packed layer, unnecessary release of microorganisms in the upper-stage packed layer can be prevented and clogging of the lower-stage packed layer can be prevented. Accordingly, the ability of the biological desulfurization apparatus to remove hydrogen sulfide can be stabilized over a long period of time.

The apparatus in the second aspect of the invention is constituted preferably such that the upper-stage aeration unit and the lower-stage aeration unit are provided respectively with an aeration pipe having a plurality of air discharging openings formed therein, and the aeration pipe is arranged along the angle of each of the supporting plates for supporting the upper- and lower-stage packed layers, and the discharging openings of the aeration pipe are formed so as to face the opposite side of the angle. By such constitution, the blockage of water and gas flow paths by the aeration pipe is reduced. By forming the aeration pipe with discharging openings on the downside, carrier-adhering matter dropped from the upper-stage packed layer (or the lower-stage packed layer) can be prevented from entering through the discharging openings, so that the clogging of the aeration pipe can be prevented and the washing effect can be stably maintained. Accordingly, the ability of the biological desulfurization apparatus to remove hydrogen sulfide can be highly stabilized over a long period of time.

The apparatus in any of the second to fourth aspects of the invention is preferably constituted such that the water level upon filling the reaction tower with water is judged according to the temperature of the external surface of the reaction tower. That is, in the reaction tower, the temperature of the external surface of the reaction tower that is filled with water is near to the temperature of the water, whereas the temperature of the external surface of the reaction tower which is not filled with water is near to room temperature, and thus the borderline there between can be judged as the water level. It follows that by the constitution described above, the water level in the reaction tower can be easily judged to facilitate the washing operation.

According to the second to fifth aspects of the invention, the biological desulfurization apparatus provided with the two-stage packed layers can facilitate washing of the packed layers, to improve the effect of washing.

The biological desulfurization apparatus in a sixth aspect of the invention is a biological desulfurization apparatus in a flow system in which sulfur-oxidizing bacteria are allowed to adhere to a carrier in a carrier-packed layer arranged in a reaction tower, to oxidize hydrogen sulfide in biogas to a sulfur component or sulfate ion, wherein when the carrier-packed layer is clogged by adhesion of microorganisms along with time and a sulfur component, the time for efficiently washing the carrier is judged according to any of the following (a) to (f):

(a) the water level of a circulation tank, which is used at the start of the apparatus,

(b) reduction in the amount of air fed to sulfur-oxidizing bacteria,

(c) differential pressure between the front and rear of the carrier-packed layer,

(d) reduction in the rising speed of a gas holder,

(e) elevation of vibration noise of a treated-water pipe for discharging treated water from the reaction tower, and

(f) overflow from an air-bleeding valve arranged in a water seal portion arranged somewhere in the treated-water pipe.

According to the sixth aspect of the invention, the time for washing the carrier-packed layer can be appropriately determined by judging the water level in a circulation tank, reduction in the amount of air fed to sulfur-oxidizing bacteria introduced into the reaction tower, differential pressure between the front and rear of the carrier-packed layer in the reaction tower, reduction in the rise velocity of a gas holder arranged somewhere in a biogas flow pipe connected to the reaction tower, vibration noise of a treated-water pipe connected to the reaction tower, or overflow from a siphon-preventing valve arranged in the treated-water pipe.

Now, specific examples of the biological desulfurization apparatus for biogas in accordance with embodiments of the invention are described in detail with reference to the drawings. Embodiments of the invention are not limited to the following description.

### Example 1

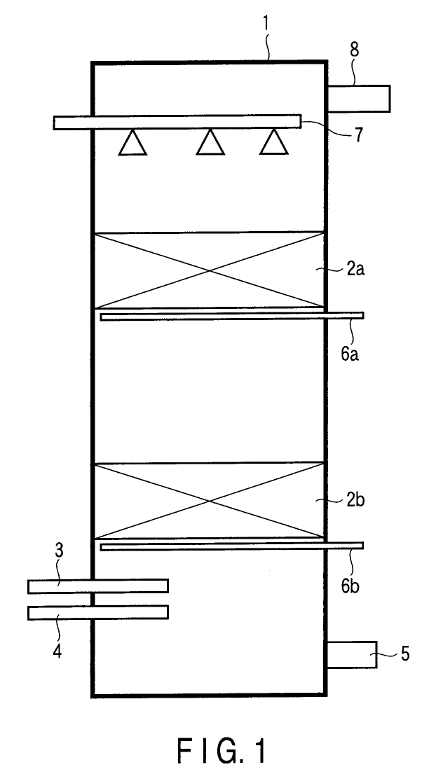

For the biological desulfurization apparatus for biogas in Example 1, FIG. 1 is referred to. Numeral 1 in the figure is a reaction tower (biological reaction tank) into which biogas generated by anaerobically fermenting organic waste is introduced. Upper-stage packed layer 2a and lower-stage packed layer 2b provided respectively with a carrier to which microorganisms had adhered are arranged vertically in the reaction tower 1. The diameter of the carrier constituting the lower-stage packed layer 2b is set greater than the diameter of the carrier constituting the upper-stage packed layer 2a. An air feeding pipe for feeding air (air feeder) 3, a biogas feeding pipe 4 for feeding biogas to the reaction tower 1, and a drain tube 5 for discharging water supplied to the reaction tower 1 are arranged respectively below the lower-stage packed layer 2b in the reaction tower 1. Washing air pipes 6a and 6b are arranged below the upper-stage packed layer 2a and lower-stage packed layer 2b respectively in the reaction tower 1. A sprinkling mechanism (sprinkling nozzle) 7 for feeding water to microorganisms in the reaction tower 1 is arranged in an upper part of the reaction tower 1. Numeral 8 in the figure refers to an outlet pipe for treated gas (treated-gas pipe) after biological desulfurization.

In the biogas biological desulfurization apparatus thus constituted, hydrogen sulfide gas contained in biogas supplied from the lower part of the reaction tower 1 is oxidatively treated by microorganisms that have adhered to the upper-stage packed layer 2a and lower-stage packed layer 2b packed in the reaction tower 1.

That is, the two-stage layers, i.e., the upper-stage packed layer (carrier-packed layer) 2a and lower-stage packed layer (carrier-packed layer) 2b are arranged in the reaction tower 1 and provided with washing air pipes 6a and 6b respectively so as to enable washing each of them, whereby only the lower part where solids are easily accumulated, or only the upper part, can be washed. Accordingly, the carrier-packed layers 2a and 2b in the reaction tower 1 can be prevented from clogging. Here, when the unclogged carrier-packed layers are washed simultaneously, the microorganisms adhered the carrier are peeled, which leads to a shortage of the microorganisms after washing. If only the clogged carrier-packed layer is washed, it is possible to prevent the deterioration in treatment performance which is caused by this shortage.

The carrier in the lower-stage packed layer 2b in which solids are heavily accumulated can be made larger than the carrier in the upper-stage packed layer 2a to prolong the period up to clogging in the lower-stage packed layer 2b. It follows that by reducing the frequency of washing, the amount of microorganisms adhering to the lower-stage packed layer 2b can be increased and the efficiency of treatment can be improved.

In Example 1, adhering microorganisms can be held in washing of the carrier-packed layer upon clogging and thus enable stable treatment as described above.

### Example 2

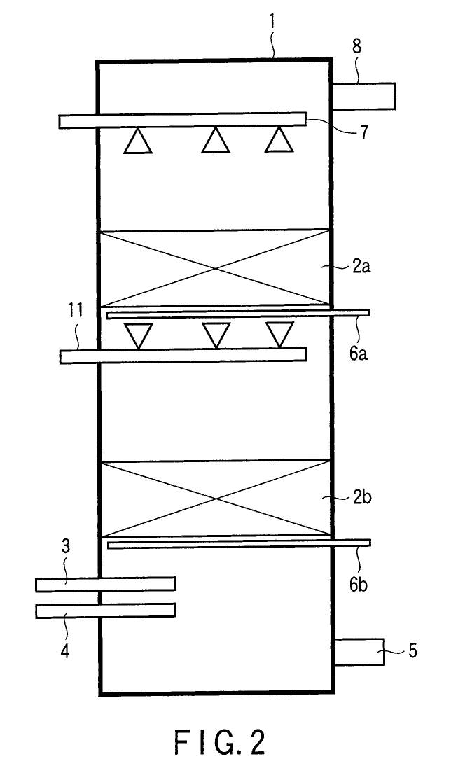

For the biological desulfurization apparatus for biogas in Example 2, FIG. 2 is referred to. The same members as in FIG. 1 are assigned like numerals to omit their description, and the main part only is described.

Numeral 11 in the figure is an intermediate sprinkling mechanism (sprinkling nozzle) arranged to face upward in the reaction tower 1 between the upper-stage packed layer 2a and lower-stage packed layer 2b.

In the biological desulfurization apparatus for biogas in FIG. 2, it is possible to feed water not only from the sprinkling mechanism 7 arranged in an upper part of the reaction tower 1, but also from the intermediate sprinkling mechanism 11. As described in Example 1, hydrogen sulfide gas contained in biogas flowing from the biogas feeding pipe 4 in the lower part of the reaction tower 1 is oxidized by microorganisms adhering to the surfaces of the upper-stage packed layer 2a and lower-stage packed layer 2b and thereby precipitated as a sulfur component. As sprinkling water falls, the sulfur component formed on the surface of the upper-stage packed layer 2a drops into the lower-staged packed layer 2b.

Both the sulfur component formed on the surface of the lower-stage packed layer 2b and the sulfur component dropping from the upper-stage packed layer 2a are accumulated in the lower-stage packed layer 2b, and thus the amount of solids accumulated in the lower-stage packed layer 2b is increased. In such case, when the flow rate from the sprinkling mechanism 7 is increased to prevent solids from accumulating in the lower-stage packed layer 2b, the microorganisms that have adhered to the surface of the upper-stage packed layer 2b also flow, thus resulting in a reduction in the amount of the microorganisms in the reaction tower, so the performance of the apparatus in biological desulfurization may be deteriorated.

In Example 2, however, sprinkling water is additionally introduced from the intermediate sprinkling mechanism 11 arranged between the upper-stage packed layer 2a and lower-stage packed layer 2b, thereby enabling washing and elimination of solids accumulated in the lower-stage packed layer 2b even more. At this time, it is predicted that the microorganisms that have adhered to the surface of the lower-stage packed layer 2b also flow out, but a rapid drop in performance can be prevented by replenishment with microorganisms dropping from the upper-stage packed layer 2a.

The intermediate sprinkling mechanism 11 is arranged to face upward, thereby showering the upper-stage packed layer 2a directly with water to wash the supporting plate for the upper-stage packed layer 2a and to prevent clogging of the upper-stage packed layer 2a as well.

According to Example 2, clogging of the upper-stage packed layer 2a can be prevented by sprinkling, and simultaneously, adhering microorganisms can be maintained, thus achieving stable treatment, as described above.

### Example 3

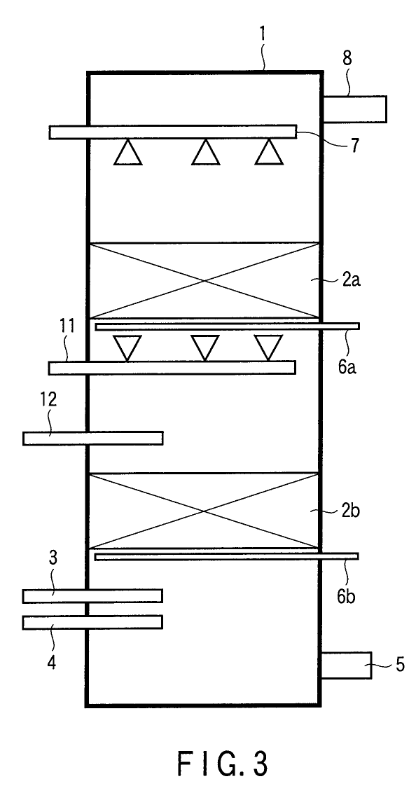

For the biological desulfurization apparatus for biogas in Example 3, FIG. 3 is referred to. The same members as in FIGS. 1 and 2 are assigned like numerals to omit their description, and the main part only is described.

Numeral 12 in the figure is an intermediate air feeding pipe (hollow air feeder) for feeding air arranged between the upper-stage packed layer 2a and lower-stage packed layer 2b.

In the biological desulfurization apparatus for biogas in FIG. 3, it is possible to feed air not only from an air feeding pipe 3 arranged in the lower part of the reaction tower 1, but also from the intermediate air feeding pipe 12. The microorganisms that have adhered to the surface of the carrier in the upper-stage packed layer 2a and lower-stage packed layer 2b require oxygen for their growth because they are aerobic, sulfur-oxidizing bacteria of the genus Thiobacillus. When the air feeding pipe 3 is present at only the same site as in the biogas inflow portion and the concentration of hydrogen sulfide in biogas fluctuates, or when the amount of microorganisms that have adhered to the surface of the carrier in the lower-stage packed layer 2b at the upstream side of gas inflow is increased too much, oxygen fed through the air feeding pipe 3 may be consumed by the microorganisms, to make it impossible to supply the oxygen necessary for inhabitation of the microorganisms that have adhered to the surface of the carrier in the upper-stage packed layer 2a at the downstream side.

However, air is supplied through the intermediate air feeding pipe 12 between the divided layers, that is, the upper-stage packed layer 2a and lower-stage packed layer 2b, whereby oxygen can be supplied to the microorganisms that have adhered to the surface of the carrier in the upper-stage packed layer 2a at the downstream side, even when the concentration of hydrogen sulfide contained in influent biogas fluctuates or when the amount of the microorganisms that have adhered to the surface of the carrier in the lower-stage packed layer 2b increases. Accordingly, the activity of the microorganisms can be maintained, and even if the concentration of hydrogen sulfide in influent biogas fluctuates, the efficiency of treatment can be stabilized.

In Examples 1 to 3 described above, washing air is introduced, for example, through two washing air pipes into the reaction tower, but this is not restrictive; alternatively, both the washing air pipes may be connected to a manifold to introduce washing air from the pipes simultaneously into the reaction tower.

### Example 4

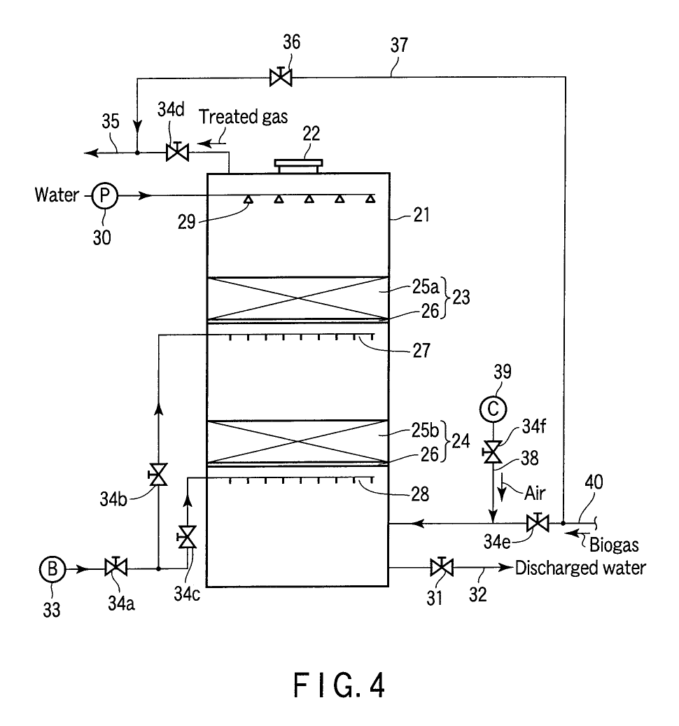

FIG. 4 is referred to. Numeral 21 in the figure is a biological desulfurization tower (reaction tower) provided in the upper part with a lid 22 capable of opening and closing. Upper-stage packed layer 23 and lower-stage packed layer 24 are arranged vertically in the reaction tower 21 respectively. The upper-stage packed layer 23 is constituted of an upper-stage carrier 25a and a supporting plate 26 for supporting the upper-stage carrier 25a. The lower-stage packed layer 24 is constituted of a lower-stage carrier 25b and a supporting plate 26 for supporting the lower-stage carrier 25b.

An upper-stage aeration unit 27 and lower-stage aeration unit 28 are arranged below the upper-stage packed layer 23 and lower-stage packed layer 24 respectively. A shower nozzle 29 is arranged above the upper-stage packed layer 23 in the reaction tower 21, and water is jetted from the shower nozzle 29 by pump 30. A treated-water pipe 32 having a drain valve 31 interposed therein is connected to a lower part of the reaction tower 21. The upper-stage aeration unit 27 and lower-stage aeration unit 28 are supplied with air via valves 34a, 34b and 34c by a blower 33.

To introduce biogas into the reaction tower 21, a biogas inlet tube 40 having valve 34e interposed therein is connected to the base of the reaction tower 21. A pipe 38 having valve 34f interposed therein is connected somewhere to the biogas inlet tube 40. Air necessary for oxidative decomposition of hydrogen sulfide in biogas is sent to the biogas inlet tube 40 by compressor 39. To discharge a treated gas in the reaction tower 21, a pipe 35 having valve 34d interposed therein is connected to an upper part of the reaction tower 21. The inlet side of the biogas inlet tube 40 and the outlet side of the pipe 35 are connected via a bypass pipe 37 having a bypass valve 36 interposed therein. When the biological desulfurization apparatus is washed, valve 34e and valve 34d are closed, valve 36 is opened, and biogas is passed through the bypass pipe 37 and thereby bypassed through the biological desulfurization apparatus.

In the biological desulfurization apparatus thus constituted, the lid 22 is opened, the drain valve 31 is closed, and the upper-stage packed layer 23 is filled with water above it, and in this state, the upper-stage packed layer 23 is washed with aeration by feeding air to an upper-stage aeration unit 27. Thereafter, the upper-stage aeration unit 27 is stopped, the drain valve 31 is opened, and the water level is reduced to a level below the upper-stage packed layer 23 and above the lower-stage packed layer 24, and in this state, the lower-stage packed layer 24 is washed with aeration by feeding air to a lower-stage aeration unit 28. As the water, secondary effluent from effluent treatment facilities for example is utilized.

More specifically, the drain valve 31 is closed, and by pump 30, water is jetted to the reaction tower 21, thereby filling the inside of the reaction tower 21 with water. By aeration with the upper-stage aeration unit 27 arranged below the upper-stage packed layer 23, the carrier in the upper-stage packed layer 23 moves due to the buoyancy of air bubbles, to release adhering matter from the carrier. At this time, the air supplied by aeration is discharged from an opening of the reaction tower 21, so the internal pressure of the reaction tower 21 will not be increased.

When the upper-stage packed layer 23 is washed, the adhering matter released from the carrier is accumulated in the lower-stage packed layer 24, and thus the washing of the upper-stage packed layer 23 is followed by washing of the lower-stage packed layer 24, thereby enhancing the effect of washing. Further, the time required for the washing operation can be reduced with less trouble in opening and closing of valves.

According to the biological desulfurization apparatus in accordance with Example 4, the upper-stage packed layer 23 and lower-stage packed layer 24 in the biological desulfurization apparatus can be washed with minimum trouble, and simultaneously the effect of washing can be enhanced.

Although the washing frequency of the upper-stage packed layer 23 and lower-stage packed layer 24 was not referred to in Example 4, a new effect can be attained by washing under the following conditions for example. That is, in the biological desulfurization apparatus in FIG. 4, the lid 22 is opened, the drain valve 31 is closed, the lower-stage packed layer 24 is filled with water above itself by shower nozzle 29, and in this state, the lower-stage packed layer 24 only is washed by aeration with the lower-stage aeration unit 28, wherein the lower-stage packed layer 24 is washed at shorter intervals than the upper-stage packed layer 23. By so doing, the ability of the biological desulfurization apparatus to remove hydrogen sulfide can be stabilized over a long period of time.

The reason that such effect can be obtained is as follows: That is, hydrogen sulfide is changed to sulfuric acid or a sulfur component via desulfurization reaction. Sulfuric acid is discharged along with discharged water, while the sulfur component adheres to the carrier and causes clogging of the upper-stage packed layer 23 and lower-stage packed layer 24. Because it is known from previous research results that the sulfur component is formed in a larger amount in the lower-stage packed layer 24, when the lower-stage packed layer 24 is washed at a higher frequency than the upper-stage packed layer 23, unnecessary release of microorganisms in the upper-stage packed layer 23 can be prevented and clogging of the lower-stage packed layer 24 can be prevented.

To stabilize the ability of the biological desulfurization apparatus to remove hydrogen sulfide, it is preferable according to inventors' experiments that the lower-stage packed layer 24 be washed either once every 10 days or once a week, while the upper-stage packed layer 23 is washed once a month. Such frequency of washing is judged to be suitable because, in consideration of the fact that the amount of the sulfur component that has adhered to the lower-stage packed layer 24 is 4 to 5 times as large as that of the upper-stage packed layer 23, the above frequency is in conformity to the amount of the formed sulfur component. It is excessive for the time of aeration by feeding air in washing to exceed 1 minute, and when the time is 1.5 minutes, the ability of the apparatus to remove hydrogen sulfide was decreased. A time of 30 seconds was sufficient for washing. That is, slight, free movement of the carrier was considered sufficient for releasing the sulfur component from the packed layer. As a matter of course, the conditions for washing the lower-stage packed layer 24 and upper-stage packed layer 23 are not limited to the frequency and time mentioned above.

### Example 5

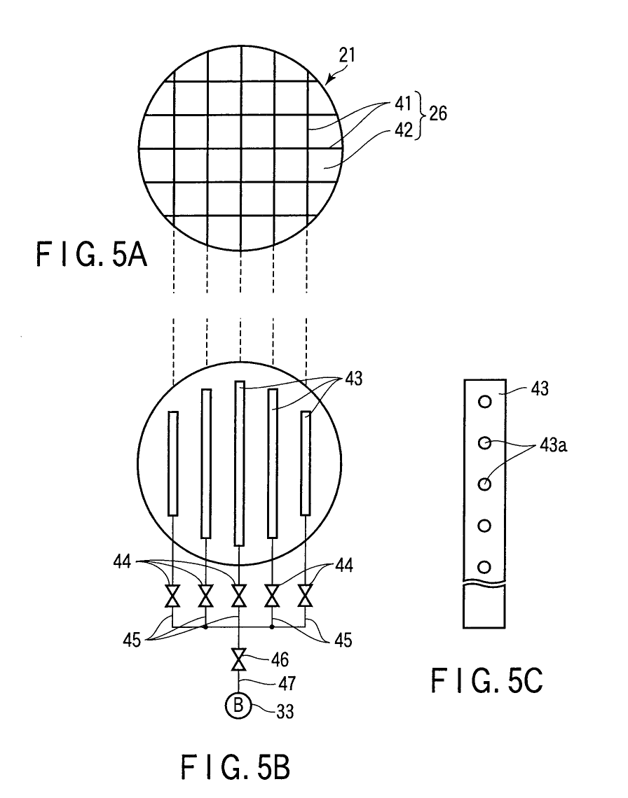

FIG. 5A, FIG. 5B and FIG. 5C are referred to. FIG. 5A is a schematic plane view of a supporting plate for the upper-stage packed layer in one constitution of the biological desulfurization apparatus. FIG. 5B is a schematic plane view showing the relationship between pipes and valve or a blower in one constitution of the upper-stage aeration unit arranged below the upper-stage packed layer. FIG. 5C is an enlarged partial back-side view of the aeration pipe in FIG. 5B. The same members as in FIG. 4 are assigned like numerals to omit their description. Description of the lower-stage packed layer is also omitted because it has the same constitution as the upper-stage packed layer.

The supporting plate 26 is composed of angles 41 arranged approximately evenly lengthwise and crosswise, and punching metals 42 arranged on the angles 41. A plurality of aeration pipes 43 constituting the upper-stage aeration unit 27 are arranged along the angle 41 in one direction (vertical direction in FIG. 5A) in the upper part of the supporting plate 26. Air discharging openings 43a are formed in the aeration pipe 43, so as to face the opposite direction of the angle 41. Pipes 45 each having valve 44 interposed therein are connected to aeration pipes 43 respectively. The pipes 45 are connected to blower 33 via pipe 47 having valve 46 interposed therein.

According to Example 5 above, the aeration pipe 43 is arranged along the angle 41 of the supporting plate 26, thereby reducing the blockage of water and gas flow paths by the aeration pipe 43. By forming the aeration pipe 43 with discharging openings 43a on the downside, carrier-adhering matter dropped from the upper-stage packed layer 23 (or the lower-stage packed layer 24) can be prevented from entering into discharging openings 43a, so the aeration pipe 43 can be prevented from clogging, and the washing effect can be stably maintained. Accordingly, the ability of the biological desulfurization apparatus to remove hydrogen sulfide can be highly stabilized over a long period of time.

### Example 6

FIG. 4 is referred to. In Example 6, the lid 22 is opened, the drain valve 31 is closed, the supporting plate 26 of the upper-stage packed layer 23 (or of the lower-stage packed layer 24) is filled with, and immersed in, water, and then the supporting plate 26 of the upper-stage packed layer 23 (or of the lower-stage packed layer 24) is washed by aeration with the upper-stage aeration unit 27 (or the lower-stage aeration unit 28).

According to Example 6, the supporting plate 26 of the upper-stage packed layer 23 (or of the lower-stage packed layer 24) can be filled with and immersed in water to wash the supporting plate 26. At this time, most of the carrier is not immersed in water, and therefore, no or little matter adhering to the carrier is released by water. Accordingly, the supporting plate 26 only can be washed over a long time, and thus the clogging of the supporting plate 26 by products can be prevented, and the ability of the biological desulfurization apparatus to remove hydrogen sulfide can be highly stabilized over a long period of time.

### Example 7

FIG. 6 is referred to. The same members as in FIG. 4 are assigned like numerals to omit their description.

Numeral 51 in the figure is a circulation unit. The circulation unit 51 has a function of sprinkling, from the upper part of the upper-stage packed layer 23, a part of bottom accumulated water 52 dropped via the upper-stage packed layer 23 and lower-stage packed layer 24 to the bottom of the reaction tower 21 and accumulated therein. The circulation unit 51 is constituted of a pipe 53 connected to the upper and lower parts of the reaction tower 21, a valve 54 interposed in the pipe 53, a circulation water tank 55, a circulation pump 56, and a liquid sprinkling pipe 57 connected to the pipe 53.

In the biological desulfurization apparatus thus constituted, when microorganisms in the upper-staged packed layer 23 are excessively released by washing, the amount of the microorganisms in the upper-stage packed layer 23 is recovered by simultaneous use of circulation of bottom accumulated water 52 by the circulation unit 51 and water sprinkling by the shower nozzle 29.

In the biological desulfurization apparatus in FIG. 6, the microorganisms released from the upper-stage packed layer 23 drop to the lower-stage packed layer 24, and thus the lower-stage packed layer 24 is supplied with the microorganisms from the upper-stage packed layer 23. On the other hand, the upper-stage packed layer 23 is not supplied from anywhere and thus requires a longer time until the microorganisms grow than in the lower-stage packed layer 24. In Example 4, however, when the microorganisms in the upper-stage packed layer 23 are released excessively by washing, the amount of the microorganisms in the upper-stage packed layer 23 is recovered by simultaneous use of the circulation of bottom accumulated water 52 by the circulation unit 51 and water sprinkling by the shower nozzle 29. Hence, the upper-stage packed layer 23 is sprinkled with the bottom accumulated water 52 by the circulation unit 51, whereby the microorganisms released from the lower-stage packed layer 24 can be supplied to the upper-stage packed layer 23, so the amount of the microorganisms in the upper-stage packed layer 23 can be increased in a short time. Accordingly, the ability of the biological desulfurization apparatus to remove hydrogen sulfide can be recovered in a short time from deterioration caused by release of the microorganisms from the carrier by excessive washing.

When the circulation of bottom accumulated water 52 by the circulation unit 51 and water sprinkling by the shower nozzle 29 are simultaneously used, the amount of water to be sprinkled is desirably reduced. By doing so, the amount of a liquid passing through the upper-stage packed layer 23 and lower-stage packed layer 24 can be prevented from becoming excessive, thereby preventing the amount of released microorganisms from increasing, and at least the amount of bottom accumulated water 52 to be circulated is preferably deducted.

### Example 8

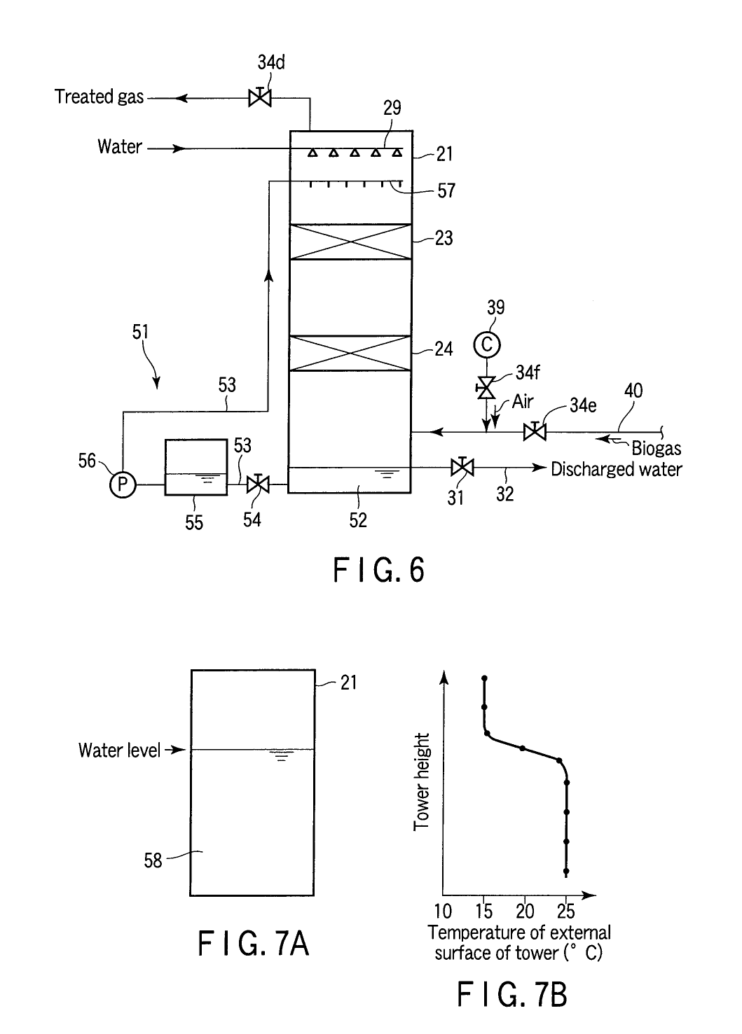

FIG. 7A and FIG. 7B are referred to. FIG. 7A is a schematic explanatory diagram of the biological desulfurization apparatus in Example 8, and FIG. 7B is a characteristic curve showing the relationship between the tower height of the water level in the reaction tower of the biological desulfurization apparatus and the temperature of the external surface of the tower. The same members as in FIG. 4 are assigned like numerals to omit their description.

The biological desulfurization apparatus in Example 8 is characterized in that the water level upon filling the reaction tower 21 with water 58 is judged according to the temperature of the external surface of the reaction tower 21. Although a thermometer can be used in measurement of the temperature, infrared thermography or the like can be used to judge the water level more easily. In the apparatus of such constitution, the temperature of that external surface of the reaction tower 21 which is filled with water 58 is near to the water temperature, while the temperature of that external surface of the reaction tower 21 which is not filled with water is near to the room temperature (see FIG. 7B). Accordingly, the borderline therebetween can be judged as the water level. According to Example 8, the water level in the reaction tower 21 can thus be easily judged to facilitate the washing operation.

### Example 9

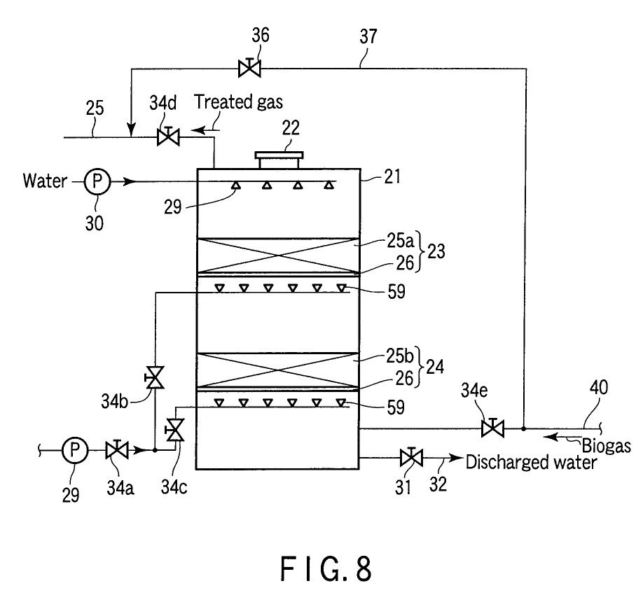

FIG. 8 is referred to. The same members as in FIG. 4 are assigned like numerals to omit their description.

Numeral 59 in the figure is a high-pressure washing nozzle of a high-pressure washing unit, and the nozzles are arranged below the upper-stage packed layer 23 and lower-stage packed layer 24, respectively. In the biological desulfurization apparatus in FIG. 8, the high-pressure washing nozzle 59 of a high-pressure washing unit is supplied with water, thereby washing the upper-stage packed layer 23 and lower-stage packed layer 24.

According to Example 9, matter adhering to the supporting plate 26 for the upper-stage packed layer 23 and lower-stage packed layer 24 can be released by jetting pressurized water from the lower side of the supporting plate 26. By moving the carrier by water pressure, the release of adhering matter from the carrier can be facilitated. As a result, the supporting plate 26 can be washed in a short time without the necessity for filling the reaction tower 21 with water, and the short time of the biological desulfurization apparatus for washing can be reduced. According to Example 9, the upper-stage packed layer 23 and lower-stage packed layer 24 in the biological desulfurization apparatus can be washed in a short time, and the ability of the biological desulfurization apparatus to remove hydrogen sulfide can be highly stabilized over a long period of time.

In Examples 4 to 9 above, a lid capable of opening and closing is arranged in the upper part of the reaction tower, but this is not restrictive; for example, a valve may be arranged in the upper part of the reaction tower.

### Example 10

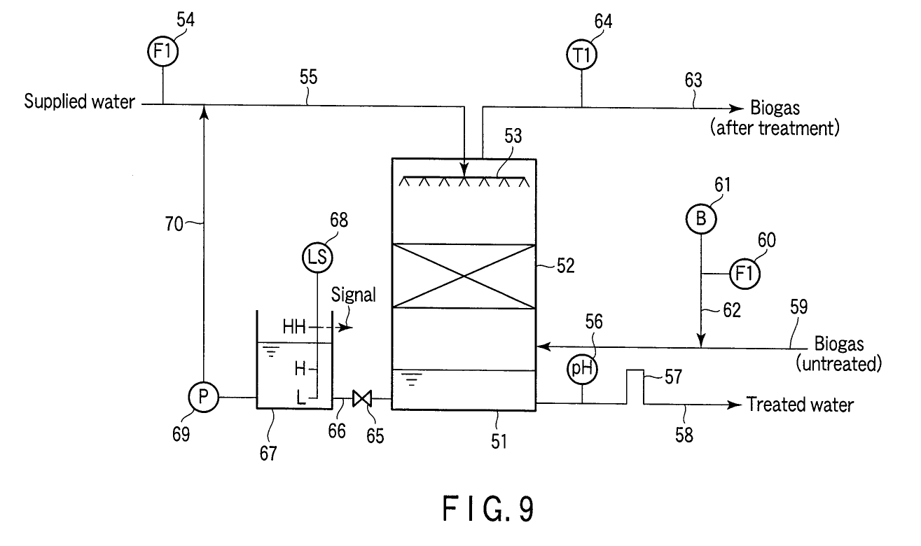

The biological desulfurization apparatus of the present invention in Example 10 is described in detail with reference to FIG. 9. Numeral 51 in the figure refers to a reaction tower in which a carrier-packed layer 52 is arranged. A sprinkling nozzle 53 is arranged in an upper part of the reaction tower 52. A water feeding pipe 55 having a flowmeter 54 interposed therein is connected to the sprinkling nozzle 53. Water is supplied from the water feeding pipe 55 via the sprinkling nozzle 53 to microorganisms. The flow rate in feeding water is controlled with the flowmeter 54.

A treated-water pipe 58 having a pH meter 56 interposed therein and having a water seal portion 57 is connected to a lower part of the reaction tower 51. Treated water is discharged via the water seal portion 57 from the treated-water pipe 58. The pH of discharged water is controlled with a pH meter 56. A biogas inflow pipe 59 for feeding biogas (untreated) to the reaction tower is connected to a lower part of the reaction tower 51. An air introducing pipe 62 having an airflow meter 60 interposed therein and connected to blower 61 is connected at some point to the biogas inflow pipe 59. Biogas mixed with air for microbial growth sent from blower 61 is introduced into the reaction tower 51. The air flow is controlled by an airflow meter 60.

A biogas flow pipe 63 for draining off biogas after treatment is connected to the upper part of the reaction tower 51. The biogas flow pipe 63 has a thermometer 64 interposed therein. The biogas that has flowed from the biogas inflow pipe 59 to the reaction tower 51 contacts, and passes upward through, the carrier-packed layer 52, and then passes in a hydrogen sulfide-free state through the biogas flow pipe 63, followed by delivery to facilities where the treated gas is effectively utilized. The temperature suitable for the microorganisms is controlled with the thermometer 64.

Circulation tank 67 is connected to a lower part of the reaction tower 51 via a communicating pipe 66 having a valve 65 interposed therein. A water level indicator 68 for measuring the water level in the circulation tank 67 is arranged in the circulation tank 67. The circulation tank 67 and water feeding pipe 55 are connected via a pipe 70 having a circulation pump 69 interposed therein. The reaction tower 51, the circulation tank 67 and the circulation pump 69 are arranged in a loop state with the communicating pipe 66, the pipe 70 and the water feeding pipe 55. The circulation tank 67 is used to fix microorganisms at the start of the apparatus, and discharged water for the start of the apparatus is sprinkled in the reaction tower 51 by the circulation pump 69.

In the biological desulfurization apparatus thus constituted, the circulation tank 67, the circulation pump 69 and the valve 65 are used at the start of the apparatus. That is, the operation of the circulation pump 69 at the start of the apparatus is conducted in the H-L level by the water level indicator 68 arranged in the circulation tank 67, but during steady operation, the circulation pump 69 is stopped, and an operation of pouring water (flowing operation) is conducted. When the carrier-packed layer 52 in the reaction tower 51 is clogged, the pressure of biogas at the inlet side is increased. If left unattended, the treated water pipe 58 is vibrated and eventually the water seal portion 57 is broken, and untreated biogas flows out.

In the present invention, the valve 65 is opened even during steady operation, and when pressure is increased due to clogging from usual pressure (about 200 to 300 mmAq), the level upon pressure before the height of the water seal portion 57 (about 500 mmAq) is assumed as HH and indicated as a signal of washing time or indicated on a control panel.

According to Example 10, the water level indicator 68 for measuring the water level in the circulation tank is arranged in the circulation tank 67 among the reaction tower 51, the circulation tank 67 and the circulation pump 69 that are arranged in a loop state with the communicating pipe 65, the pipe 70 and the water feeding pipe 55, and when the carrier-packed layer 52 is clogged by adhesion of microorganisms along with time and the sulfur component, the time for efficiently washing the carrier is indicated as a signal of washing time or indicated on a control panel, and can thus be judged according to the water level of the circulation tank 67 used at the start of the apparatus.

In determining the suitable time for washing the carrier by detection of the water level in the circulation tank 67 in Example 1, the originally arranged circulation tank 67 and water level indicator 68 can be used as described above to determine the washing time easily and accurately. Accordingly, this method is a very gentle and accurate detection method wherein the increase in the internal gas pressure of the reaction tower 51 is converted into a change in the water level of the circulation tank 67, so this method can be said to be a monitoring method very adapted to environmental plants such as the present facilities.

### Example 11

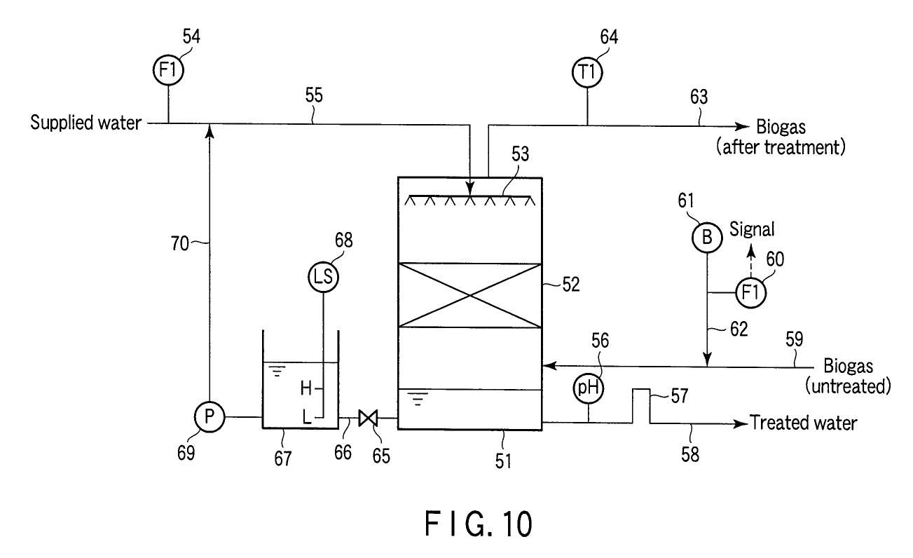

The washing method of the carrier of the biological desulfurization apparatus of the present invention in Example 11 is described in detail with reference to FIG. 10. The same members as in FIG. 9 are assigned like numerals to omit their description, and the main part only is described.

The apparatus in Example 11 is characterized in that when the carrier-packed layer 52 is clogged by adhesion of microorganisms along with time and the sulfur component, the time for efficiently washing the carrier can be judged according to reduction in the amount of air fed to sulfur-oxidizing bacteria.

In FIG. 10, the air flow of blower 61 is lower than the air flow of biogas, and thus the air flow is decreased by an increase in the pressure of biogas at the inlet side. By utilizing this phenomenon, the time when the flow rate of airflow meter 60 reaches about 80% of the rated flow is indicated as a signal of washing time on a control panel.

By utilizing the phenomenon in which the air flow is decreased by an increase in the pressure of biogas at the inlet side according to Example 11, the time when the flow rate of the airflow meter 60 reaches about 80% of the rated flow is indicated as a signal of washing time on a control panel, whereby the time for efficiently washing the carrier can be appropriately judged.

### Example 12

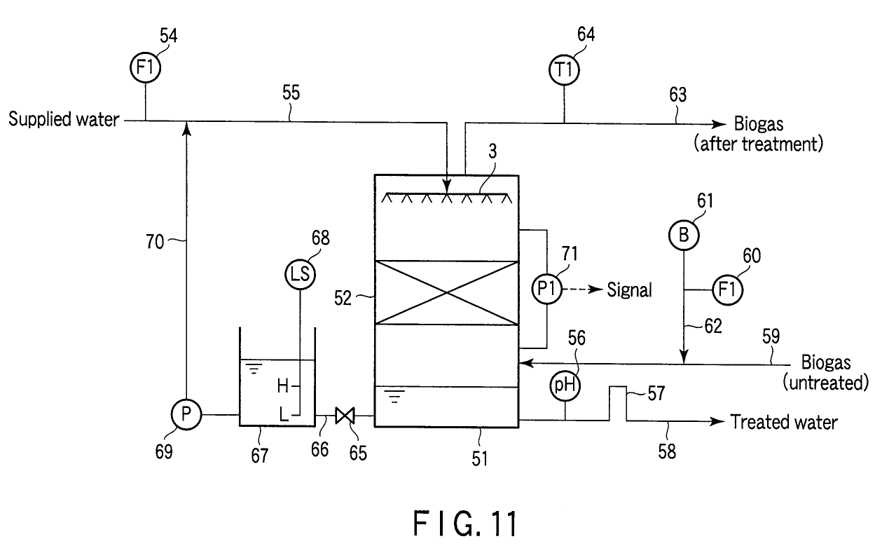

The biological desulfurization apparatus of the present invention in Example 12 is described in detail with reference to FIG. 11. The same members as in FIG. 9 are assigned like numerals to omit their description, and the main part only is described.

The apparatus in Example 12 is characterized in that when the carrier-packed layer 52 is clogged by adhesion of microorganisms along with time and the sulfur component, the time for efficiently washing the carrier can be judged according to the differential pressure between the front and rear of the carrier-packed layer. Numeral 71 in the figure is a differential pressure gauge for detecting the differential pressure between the front and rear of the carrier-packed layer 52. Specifically, the differential pressure between the front and rear of the carrier-packed layer 52, when detected at about 200 mmAq with the differential pressure gauge 71, is indicated as a signal of washing time on a control panel.

According to Example 12, the differential pressure between the front and rear of the carrier-packed layer 52 is detected with the differential pressure gauge 71, and a signal of washing time based thereon is indicated on a control panel, whereby the time for efficiently washing the carrier can be appropriately judged.

### Example 13

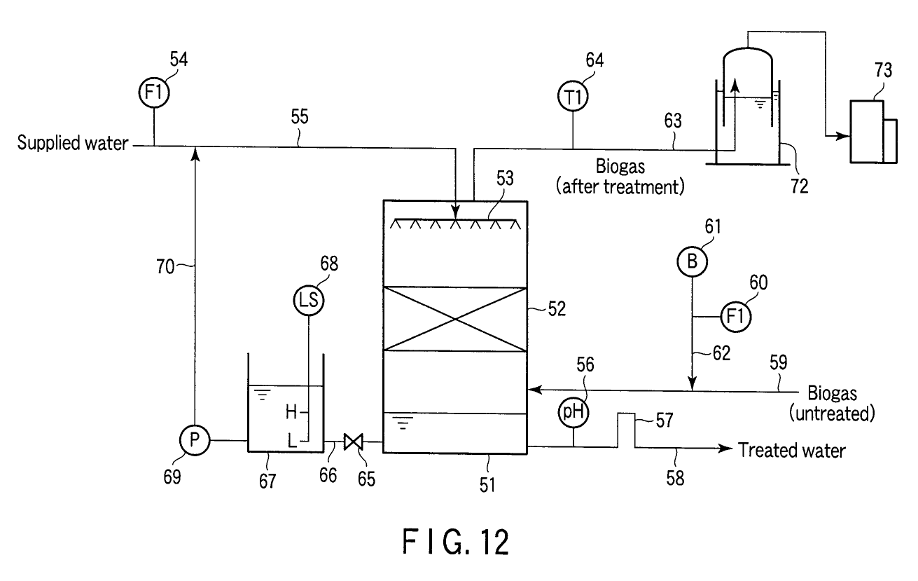

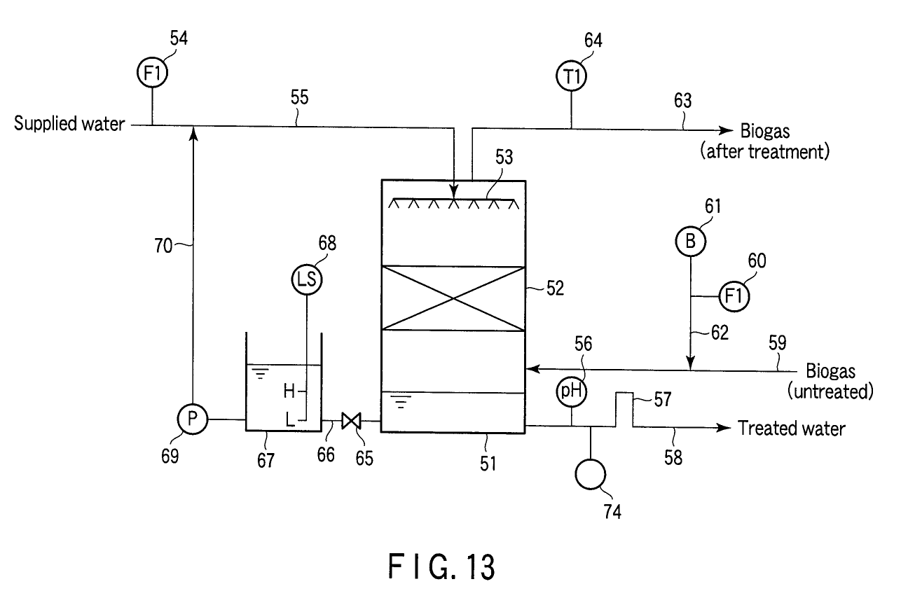

The biological desulfurization apparatus of the present invention in Example 13 is described in detail with reference to FIG. 12. The same members as in FIG. 9 are assigned like numerals to omit their description and the main part only is described.

The apparatus in Example 13 is characterized in that when the carrier-packed layer 52 is clogged by adhesion of microorganisms along with time and the sulfur component, the time for efficiently washing the carrier can be judged according to reduction in the rise velocity of a gas holder. Numeral 72 in the figure refers to a gas holder arranged at some point in the biogas flow pipe 63, and numeral 73 refers to a gas boiler. When the carrier-packed layer 52 is clogged, the amount of biogas after treatment is reduced, and thus the rise velocity of the gas holder 72 is reduced. In Example 4, this phenomenon is detected by calculation using operating data of the gas boiler, to wash the carrier-packed layer 52.

According to Example 13, the time for efficiently washing the carrier can be appropriately judged in this manner by the gas holder 72 arranged at some point in the biogas flow pipe 63.

### Example 14

The biological desulfurization apparatus of the present invention in Example 14 is described in detail with reference to FIG. 13. The same members as in FIG. 9 are assigned like numerals to omit their description, and the main part only is described.

The apparatus in Example 14 is characterized in that when the carrier-packed layer 52 is clogged by adhesion of microorganisms along with time and the sulfur component, the time for efficiently washing the carrier can be judged according to elevation of vibration noise of a treated-water pipe 58 for discharging treated water from the reaction tower 51. Numeral 74 in the figure refers to a vibration meter arranged somewhere in the treated-water pipe 58. That is, when the carrier-packed layer 52 is clogged by adhesion of microorganisms along with time and the sulfur component, the pressure of biogas at the inlet side is increased, to cause vibration of the treated-water pipe 58. In Example 14, this vibration noise is detected with the vibration meter 74, whereby the time for efficiently washing the carrier is judged.

According to Example 14, the time for efficiently washing the carrier can be appropriately judged in this manner by the vibration meter 74 arranged at some point in the treated-water pipe 58.

### Example 15

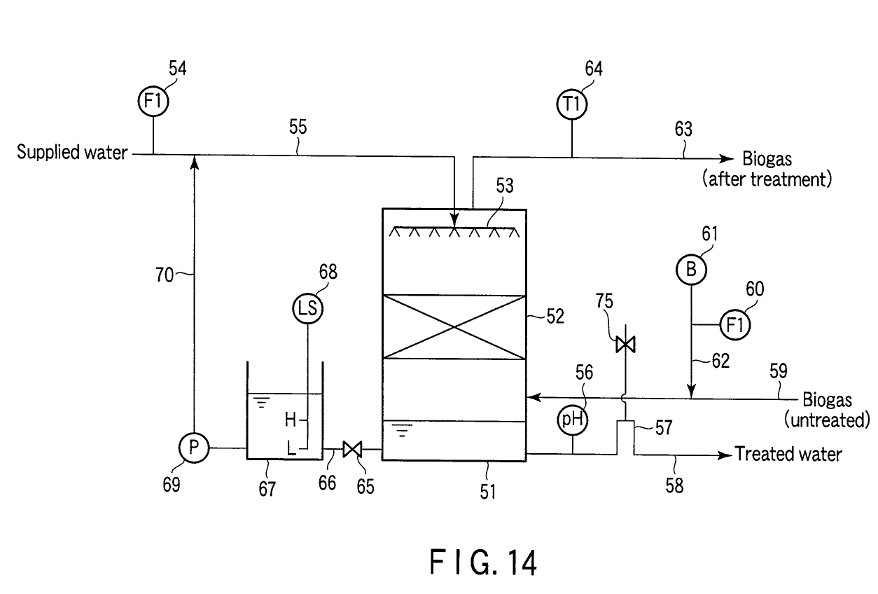

The biological desulfurization apparatus of the present invention in Example 15 is described in detail with reference to FIG. 14. The same members as in FIG. 9 are assigned like numerals to omit their description, and the main part only is described.

Numeral 75 in the figure refers to an air-bleeding valve (treated-water pipe siphon preventing valve) arranged at some point in the treated-water pipe 58. That is, when the carrier-packed layer 52 is clogged by adhesion of microorganisms along with time and the sulfur component, the pressure of biogas at the inlet side is increased to push treated water in the reaction tower 51 into the treated-water pipe 58. In Example 15, treated water is pushed into the treated-water pipe 58, provided with an air-bleeding valve 75, and overflowing treated water is detected visually as a signal for washing the carrier-packed layer 52. Usually, the water level of treated water in the reaction tower 51 is kept at the highest water level of the water seal portion 57.

According to Example 15, the time for efficiently washing the carrier can be appropriately judged in this manner by the air-bleeding valve 75 arranged at some point in the treated-water pipe 58.

1. A biological desulfurization apparatus comprising a reaction tower into which biogas generated by anaerobic fermentation of organic waste is to be introduced, a carrier-packed layer arranged in the reaction tower and provided with a carrier to which microorganisms have adhered, an air feeder that mixes air into a lower part of the reaction tower, and a sprinkling mechanism that sprinkles water on an upper part of the reaction tower, wherein the carrier-packed layer is arranged in two or more stages.

2. The biological desulfurization apparatus according to claim 1, wherein the diameter of the carrier in the carrier-packed layer at the lower-stage side is larger than the diameter of the carrier in the carrier-packed layer at the upper-stage side.

3. The biological desulfurization apparatus according to claim 1 or 2, wherein an air pipe for washing is arranged in each of the carrier-packed layers, thereby allowing the carrier-packed layers to be washed separately from one another.

4. The biological desulfurization apparatus according to claim 1 or 2, wherein a sprinkling mechanism is arranged in every carrier-packed layer or in every predetermined number of carrier-packed layers.

5. The biological desulfurization apparatus according to claim 4, wherein a sprinkling mechanism other than that in the uppermost stage is directed upward.

6. The biological desulfurization apparatus according to claim 1 or 2, wherein a plurality of carrier-packed layers are further provided in an intermediate portion with an intermediate air feeder which introduces air.

7. The biological desulfurization apparatus according to claim 4, wherein the amount of water to be sprinkled from the sprinkling mechanism nearer to the side of biogas inflow is increased.

8. A biological desulfurization apparatus comprising a reaction tower provided at the upper part thereof with a lid or valve capable of opening and closing, upper- and lower-stage packed layers arranged vertically in the reaction tower respectively and consisting of a carrier and a supporting plate for supporting the carrier, upper- and lower-stage aeration units arranged on the undersides of the upper- and lower-stage packed layers respectively, and a treated-water pipe connected to the lower part of the reaction tower and having a drain valve interposed therein,
wherein the lid or valve is opened, the drain valve is closed, the upper-stage packed layer is filled with water above the upper-stage packed layer and washed in this state with aeration by feeding air to the upper-stage aeration unit, and thereafter, the upper-stage aeration unit is stopped, the drain valve is opened, the water level is made lower than the upper-stage packed layer and higher than the lower-stage packed layer, and in this state, the lower-stage packed layer is washed with aeration by feeding air to the lower-stage aeration unit.

9. The biological desulfurization apparatus according to claim 8, comprising:
means for washing the upper and lower-stage packed layers, and
means for washing the lower-stage packed layer, in which the lid or valve is opened, the drain valve is closed, and the lower-stage packed layer is filled with water above the lower-stage packed layer and washed in this state with aeration by feeding air to the lower-stage aeration unit,
wherein the frequency of washing of the lower-stage packed layer is carried out at shorter intervals than the frequency of washing of the upper-stage packed layer.

10. The biological desulfurization apparatus according to claim 8 or 9, wherein the upper-stage aeration unit and the lower-stage aeration unit are provided respectively with an aeration pipe having a plurality of air discharging openings formed therein, and the aeration pipe is arranged along the angle of each of the supporting plates for supporting the upper- and lower-stage packed layers, and the discharging openings of the aeration pipe are formed so as to face the opposite side of the angle.

11. A biological desulfurization apparatus comprising a reaction tower provided at the upper part thereof with a lid or valve capable of opening and closing, upper- and lower-stage packed layers arranged vertically in the reaction tower respectively and consisting of a carrier and a supporting plate for supporting the carrier, upper- and lower-stage aeration units arranged on the undersides of the upper- and lower-stage packed layers respectively, and a treated-water pipe connected to the lower part of the reaction tower and having a drain valve interposed therein,
wherein the lid or valve is opened, the drain valve is closed, and the supporting plate of the upper- or lower-stage packed layer is filled with, and immersed in, water above the supporting plate of the upper- or lower-stage packed layer and washed with aeration by feeding air to the upper- or lower-stage aeration unit.

12. A biological desulfurization apparatus comprising a reaction tower, upper- and lower-stage packed layers arranged vertically in the reaction tower, a water-sprinkling shower nozzle arranged in the upper part of the reaction tower, a circulation unit that sprinkles, from the upper part of the upper-stage packed layer, a part of bottom accumulated water dropped via the upper- and lower-stage packed layers to the bottom of the reaction tower and accumulated at the bottom, and a treated-water pipe connected to the lower part of the reaction tower and having a drain valve interposed therein,
wherein when microorganisms in the upper-stage packed layer are removed excessively by washing, the amount of the microorganisms in the upper-stage packed layer is recovered by simultaneous use of circulation of the bottom accumulated water by the circulation unit and water sprinkling by the shower nozzle.

13. A biological desulfurization apparatus according to any one of claims 8 to 12, wherein the water level upon filling the reaction tower with water is judged according to the temperature of the external surface of the reaction tower.

14. A biological desulfurization apparatus comprising a reaction tower provided at the upper part thereof with a lid or valve capable of opening and closing, upper- and lower-stage packed layers arranged vertically in the reaction tower and consisting of a carrier and a supporting plate for supporting the carrier, a high-pressure washing unit having a high-pressure washing nozzle, arranged respectively on the downsides of the upper- and lower-stage packed layers, and a treated-water pipe connected to the lower part of the reaction tower and having a drain valve interposed therein,
wherein the upper- and lower-stage packed layers are washed by feeding water to the high-pressure washing nozzle of the high-pressure washing unit.

15. A biological desulfurization apparatus in a flow system in which sulfur-oxidizing bacteria are allowed to adhere to a carrier in a carrier-packed layer arranged in a reaction tower, to oxidize hydrogen sulfide in biogas to a sulfur component or sulfate ions, wherein:
when the carrier-packed layer is clogged by adhesion of microorganisms along with time and the sulfur component, the time for efficiently washing the carrier is judged according to any of the following (a) to (f):
(a) the water level of a circulation tank, which is used at the start of the apparatus,
(b) reduction in the amount of air fed to the sulfur-oxidizing bacteria,
(c) differential pressure between the front and rear of the carrier-packed layer,
(d) reduction in the rising speed of a gas holder,
(e) elevation of vibration noise of a treated-water pipe for discharging treated water from the reaction tower, and
(f) overflow from an air-bleeding valve arranged in a water seal portion arranged somewhere in the treated-water pipe.
